(19) United States
(12) Patent Application Publication
(10) Pub. No.: US 2013/0012947 A1
(43) Pub. Date: Jan. 10, 2013

(54) DOUBLE CANNULATED OSTEOTOME

(75) Inventors: Mark Ross, Hamilton (AU); Jennifer Tarrant, Pflugerville, TX (US)

(73) Assignee: ASCENSION ORTHOPEDICS, INC., Austin, TX (US)

(21) Appl. No.: 13/542,229

(22) Filed: Jul. 5, 2012

Related U.S. Application Data
(60) Provisional application No. 61/505,944, filed on Jul. 8, 2011.

(51) Int. Cl. A61B 17/16

(52) U.S. Cl. 606/84

(57) ABSTRACT

A cannulated osteotome for cutting a bone surface in which two circular depressions have been previously reamed comprising a cutting blade in the form of a continuous loop having two arcuate end surfaces and two straight lateral surfaces that depends from a body that includes two cannulae in the form of two parallel passageways that extend transversely through the body. Stops extend beyond the opposite arcuate ends of the cutting blade which determine the depth to which the cutting blade can be inserted into the surface of bone from which the circular depressions have been reamed. There is described a method of inserting an implant having a substantially flat undersurface from which two pegs protrude into a surface of a bone using such an osteotome.

This application claims priority from U.S. Provisional Application No. 61/505,944, filed Jul. 8, 2012, the disclosure of which is incorporated herein by reference.

This invention relates generally to orthopedics and more particularly to a device and method for creating a non-circular-shape depression of desired depth in a bone. It still more particularly relates to a method for preparing a glenoid surface to accept a glenoid implant, which implant has a pair of protruding posts that are received in the prepared bone.

### BACKGROUND OF THE INVENTION

Various osteotomes have been developed for cutting a bone so as to create a shaped depression into which an implant can be seated. As one example, the shoulder or glenohumeral joint, which allows for articulation between the glenoid of the scapula and the humeral head, is often repaired by the implantation into the facing surface of the glenoid of a body formed with an articular surface. When inserting such an implant, it is important that the implanted body have precise alignment so that articulation with the humeral head or with a head replacement will then function smoothly. Various devices have been developed for creating circular depressions in a bone surface, such as that of a glenoid, and examples of such can be found in U.S. Pat. No. 4,150,675, and in published U.S. Application Nos. 2006/0058809, 2006/0195194 and 2009//0270863.

Improved methods and devices for preparing a bone surface, such as that of the glenoid, for implanting a body of non-circular shape are desired.

### SUMMARY OF THE INVENTION

An improved osteotome is provided for cutting a bone surface in which two circular depressions have been previously cut, which osteotome assures precision cutting by the use of two guide-wires employed in reaming such depressions.

An improved method of inserting an implant into a bone surface is provided where two guide-wires are implanted into the surface of the bone and two circular depressions are reamed in the bone using a cutter having a cannulated shaft that slides over the respective guide-wires. Optionally, a central hole concentric with each circular depression may be simultaneously formed. A double-cannulated osteotome is then inserted over said two guide-wires to cut the bone along two straight lines tangent to the two reamed circular depressions to prepare the cavity to receive an implant having two pins that protrude from its flat undersurface.

In one particular aspect, the invention provides an osteotome for cutting a bone surface in which two circular depressions have been previously cut, which osteotome comprises a body, and a cutting blade extending from an undersurface of said body, which cutting blade has opposite arcuate ends and two substantially straight lateral sides, two cannulae in the body in the form of two parallel passageways that extend transversely through the body, which passageways have centers that are spaced apart the same distance as the centers of the acruate ends of said cutting blade, and an upper portion of said body being formed for attachment of an upwardly extending handle to said body, and said body having stops extending outwardly beyond said cutting blade, which stops determine the depth to which the cutting blade can be inserted into the surface of bone from which the circular depressions have been cut. Also provided is a kit that includes such an osteotome, 2 guide-wires, and a pin for location in juxtaposition with the bone surface to position said 2 guide-wires at desired locations.

In another particular aspect, the invention provides a method of inserting an implant into a surface of a bone, which implant has a substantially flat undersurface from which two pegs protrude and has opposite ends that are circular arcs with centers offset from each other, which method comprises the steps of: implanting two guide-wires into the surface of the bone at the centers of two circles that includes said arcs, respectively reaming two circular depressions in the bone using a cutter having a cannulated shaft that slides over the respective guide-wires and forming a central hole concentric with each said circular depression to create two cavities to receive the pegs on the undersurface of the implant, inserting a double-cannulated osteotome over said two guide-wires, which osteotome has a cutting blade that has two spaced apart opposite ends which are arcs that have radii respectively about equal to the radii of said two circles and cutting the bone at locations generally along two straight lines extending between and tangent to said two reamed circular depressions, removing the osteotome and two generally triangular segments of bone from along the straight line cuts of the osteotome, and locating the implant in the bone in which the depressions were reamed and securing it to the bone.

### BRIEF DESCRIPTION OF THE DRAWINGS

FIG. 1 is an upper perspective view of an osetotome embodying various features of the invention.

FIG. 2 is a right side view of the osteotome shown in FIG. 1.

FIG. 3 is a top plan view of the osteotome of FIG. 1 with the handle removed.

FIG. 4 is a bottom view of the osteotome of FIG. 3.

FIG. 5 is a rear view of the osteotome of FIG. 3.

FIG. 6 is a cross-sectional view taken generally along line 6-6 of FIG. 3.

FIG. 7 is a side view of a glenoid implant.

FIG. 8 is a top view of the implant of FIG. 7.

FIG. 9 is a top view of a pin guide.

### DETAILED DESCRIPTION OF THE PREFERRED EMBODIMENTS

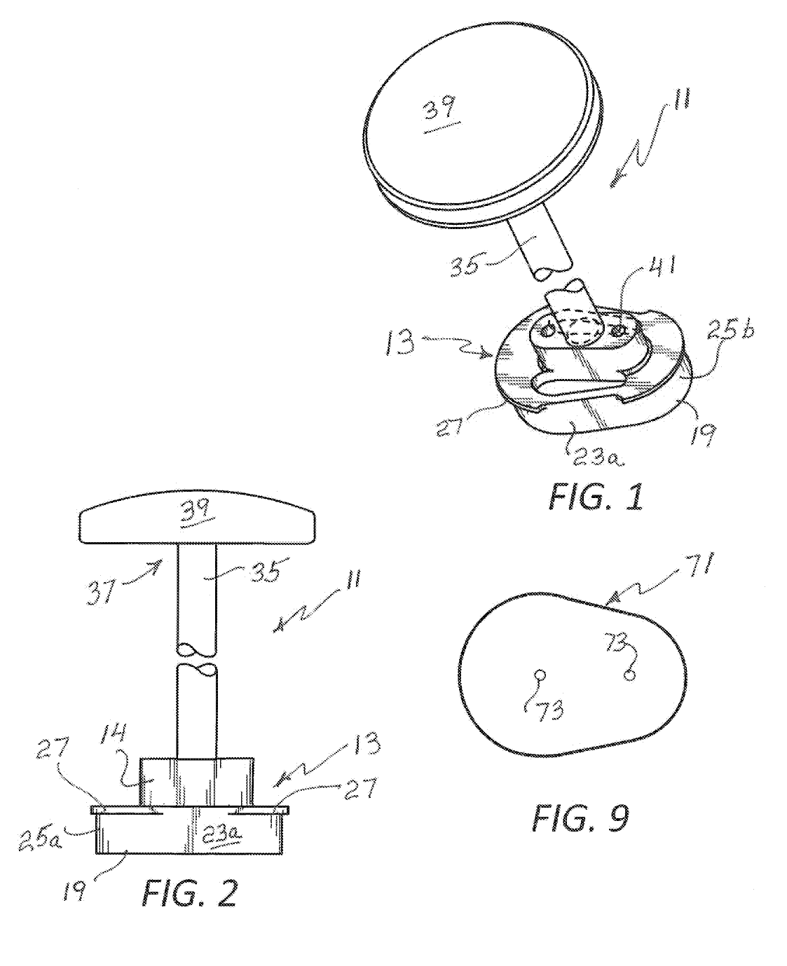

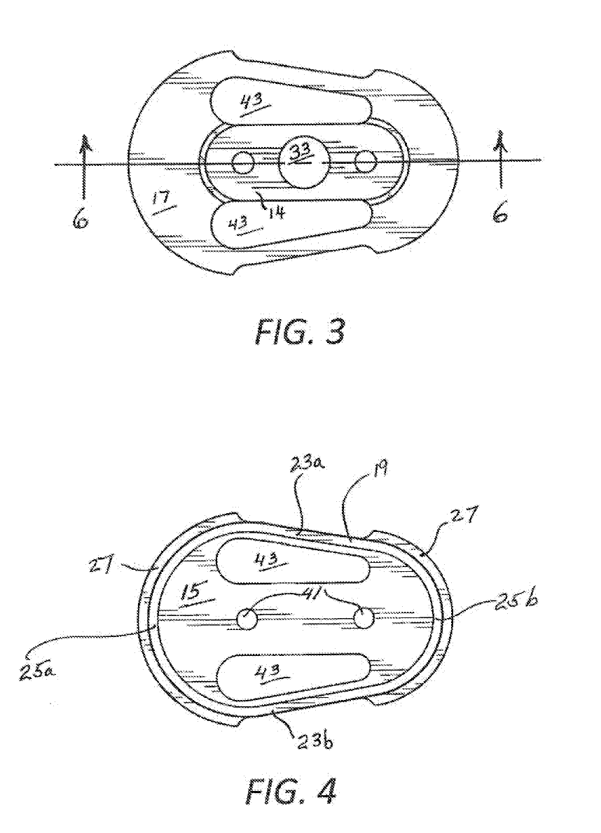

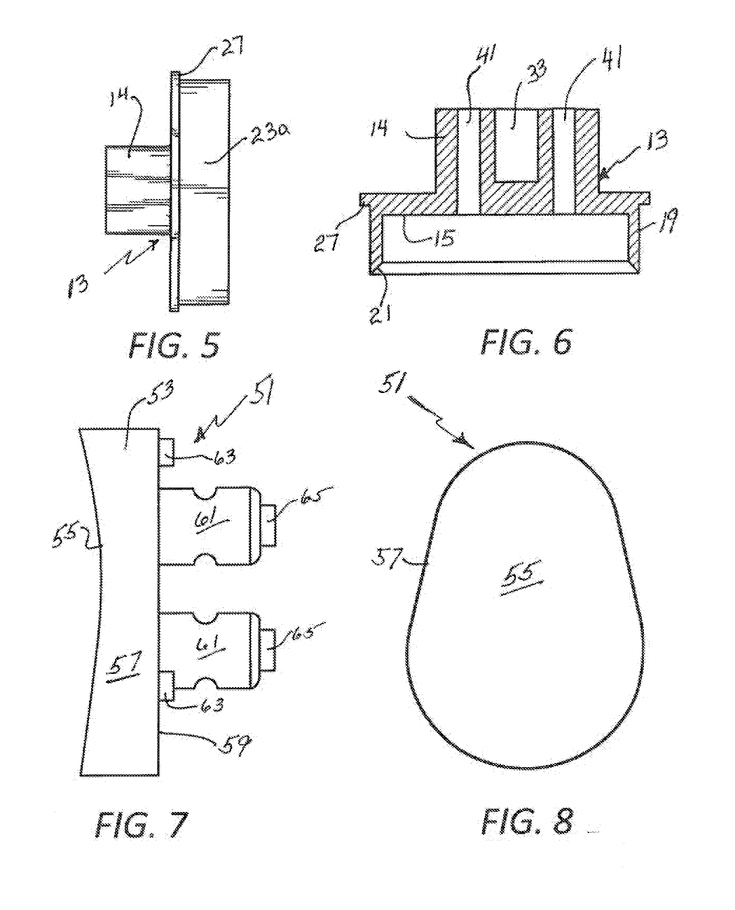

The embodiment of the invention illustrated in FIGS. 1-6 is designed to assist in the precise removal of bone from the surface of the glenoid to facilitate the insertion of a glenoid implant, such as that shown in FIGS. 7-8, that will then provide a precisely located articular surface for mating with the complementary surface of the head of the humerus or, more often, with an implant that replaces the proximal humeral head. However, it should be understood that such an osteotome could be employed in the general fashion described hereinafter to create a non-circular depression in the surface of any animal bone, such as other bones of the human skeleton.

Illustrated in FIGS. 1-6 is one embodiment of an osteotome 11 embodying various features of the invention which includes a main body 13, that comprises a generally flat undersurface 15 and a crown 14 that surmounts a generally flat upper peripheral surface 17, although the upper surface may take various other shapes if desired. A cutting blade 19 depends from the flat undersurface 15, which blade terminates in a lower cutting edge 21 that is sharpened so as to penetrate human bone. The illustrated cutting blade 19 is of rectilinear shape in the axial or vertical direction and has the form of a continuous loop, having two straight, non-parallel lateral sides 23a & 23b and two generally arcuate ends 25a & 25b that respectively join the two lateral sides at their respective ends. The acruate ends 25a,b have radii of different lengths and, as a result of their relative locations, are respectively arcs of two overlapping circles. Although the blade 19 is preferably continuous, it might be interrupted. Edge portions 27 of the body 13 form flanges that extend outward beyond the two generally arcuate ends 25a,b of the cutting blade and serve as stops that limit the depth to which cutting blade 19 can be pressed into the surface of a bone, such as the glenoid. The crown 14 is generally oval in cross-section, extending upward from the upper surface 17, and this structure includes a central cavity 33 which is designed to receive the end of an elongated stem 35 of a handle 37 having a circular head or grip 39. The handle 37 is attached to the body 13 to complete the osteotome 11 and facilitate its use in cutting bone. The handle stem 35 may simply have a distal end that is press-fit into the cavity 33, or the stem end and the cavity 33 may have mating threads. Present in the crown structure 14 are two parallel passageways or cannulae 41 of circular cross section that extend completely through the main body, opening into the flat undersurface 15 of the body. The cannulae 41 are designed to slidably receive two guide-wires, e.g. K-wires, in the form of thin stainless steel rods or pins that were earlier inserted into the bone being cut. Formed in the peripheral plate-like portion 17 of the main body 13 are a pair of oblong windows 43 which flank the central oval crown 14. The windows 43 extend through the main body 13 and provide the surgeon with a view of the center portion of the bone region below the osteotome 11 where the cutting is to occur.

The osteotome main body 13 may be machined from any suitable, strong, non-corrosive metal or metal alloy that can be sterilized for surgical use, such as stainless steels, nickel chromium alloys, and the like. The handle 37 is preferably made having a stem 35 of similar material, which might be about a quarter-inch (6-7 mm) in diameter and might have a length of about 7-8 inches (18-20 cm). The grip 39 at the upper end can be metal or any polymeric material which likewise can be sterilized. As mentioned, the distal end of the handle stem 35 can be threaded or press-fit into the corresponding cavity 33 formed in the upper surface of the crown 14 which surmounts the flat plate-like portion of the main body 13 of the osteotome.

FIGS. 7 and 8 exemplify a glenoid implant 51 for which a bone surface might be prepared for implantation using the osteotome 11 of FIGS. 1-6. The implant 51 comprises a body of 53 with a curved upper surface 55 that serves as the articular surface against which the humeral head will be engaged. The periphery 57 of the body is a rectilinear surface that has essentially the same outline as the exterior surface of blade 19 of the osetotome. An undersurface 59 of the implant 51 is flat, and there are two pegs or posts 61 which extend therefrom that have centers that are spaced apart the same distance as the centers of the two cannulae 41 in the osetotome. There are three small circular pads 63 that are carried on the flat undersurface at locations that generally flank the two pegs and similar small pads 65 on the tips of the pegs 61. These pads provide some space for cement in the region immediately between the facing surfaces of the implant and the bone cavity. The pegs 61 are of generally circular cross section but are also contoured to provide indentations that allow for cement intrusion that will assist in securing the implant 51 stably within the prepared cavity. As an example of a method of implantation of such an implant 51, once the glenoid is satisfactorily exposed by the surgeon, a suitable pin guide 71 (FIG. 9) having the outline of the implant 51 is placed at the precise desired location on the glenoid. The guide 71 has a pair of holes 73 spaced apart the same distance as the centers of the two cannulae 41 of the osteotome and the centers of the two pegs 61. A pair of guide-wires (e.g. K-wires) that may be about 2 millimeters in diameter and about 15 cm in length are driven into the glenoid until adequate purchase is achieved. The pin guide 71 is then removed, and cannulated drills and/or reamers are used to create depressions and holes to receive the pegs 61 which fit over the guide-wires. The two guide-wires assure the creation of two parallel holes of desired depth and diameter to receive the pegs 61 of the implant. The two guide-wires are respectively located at the centers of the two arcuate ends 25a, b of the cutting blade 17 which match the ends of the periphery 71 of the body of the implant. Separate cannulated drills and headed reamers are well known in the medical art and may be used. However, a cannulated combined drill/planer of appropriate size is preferably used to simultaneously drill the hole for one peg and ream a concentric circular cavity in the surface of the glenoid to the desired depths. Such devices are known in the art and can be sized to conform with the requirements for a specific implant. The drilling/reaming is then repeated about the other guide-wire using a drill/planer of desired diameter.

Once the drilling and reaming has been completed, the osteotome 11 is placed over the guide-wires which are slidably received within the cannulae 41. The osteotome 11 is then driven down with light taps from a mallet on the circular head 39 until it rests with the stops 27, that extend past the arcuate ends of the cutting blade 19, in contact with the glenoid surface. The osteotome blade 21 cuts the bone along the two lateral, non-parallel, straight sides to produce walls of the cavity in the bone that are tangent to the two arcuate end surfaces. Following removal of the osteotome and the guide-wires, bone debris is removed along with the small triangular pieces of bone that were left in the anterior and posterior aspects of the cavity which flank the region where the two circular reamers had overlapped.

With the cavity thus prepared, the glenoid implant 51 can be installed by first applying the cement to cover the underside 59 of the implant and injecting cement under pressure into each of the peg holes. With the cement in place, the implant 51 is aligned, inserted and then impacted to seat it so its front articular surface 55 sits even with the native glenoid surface. Pressure is maintained directly on the implant 51 until the cement hardens.

While the osteotome has been shown and described in accordance with the best mode developed to date by the inventors, it should be understood that various changes and modifications as would be obvious to one having ordinary skill in this art might be made to in the illustrated embodiment without deviating from scope of the invention, which is defined by the claims appended hereto. Particular features of the invention are set forth in the claims that follow.

1. An osteotome for cutting a bone surface in which two circular depressions have been previously cut, which osteotome comprises:
a body, and
a cutting blade extending from an undersurface of said body, which cutting blade has opposite arcuate ends and two substantially straight lateral sides,
two cannulae in said body in the form of two parallel passageways that extend transversely through the body, which passageways have centers that are spaced apart the same distance as the centers of the acruate ends of said cutting blade, and
an upper portion of said body being formed for attachment of an upwardly extending handle to said body, and
said body having stops extending outwardly beyond said cutting blade, which stops determine the depth to which the cutting blade can be inserted into the surface of bone from which the circular depressions have been cut.

2. The osteotome of claim 1 wherein said arcuate ends of said cutting blade have different radii and said substantially straight lateral sides are not parallel.

3. The osteotome of claim 1 wherein said cutting blade comprises a continuous loop having two arcuate end surfaces and two straight lateral surfaces.

4. The osteotome of claim 3 wherein said stops extend beyond said opposite acruate ends of said cutting blade.

5. The osteotome of claim 4 wherein said stops constitute flat flanges that extend past each respective arcuate end surface of said cutting blade and extend laterally beyond portions of said two lateral surfaces.

6. The osteotome of claim 5 wherein said generally arcuate ends of said cutting blade are positioned so as to occupy arc segments of two intersecting circles.

7. The osteotome of claim 6 wherein said body contains a pair of apertures that allow a surgeon to view the bone being cut below the osteotome.

8. A kit for preparing a bone surface to receive an implant that will be recessed at least partially below the bone surface, which kit comprises,
an osteotome according to claim 7,
2 guide-wires, and
a pin guide for location in juxtaposition with the bone surface to position said 2 guide-wires at desired locations.

9. The kit of claim 8 which further includes,
a cannulated rotary cutting head which is guidable along one of said guide-wires to ream a circular depression of desired depth and/or to cut a concentric peg hole of desired depth in the bone surface.

10. A method of inserting an implant into a surface of a bone, which implant has a substantially flat undersurface from which two pegs protrude and has opposite ends that are circular arcs with centers offset from each other, which method comprises the steps of:
implanting two guide-wires into the surface of the bone at the centers of two circles that includes said arcs,
respectively reaming two circular depressions in the bone using a cutter having a cannulated shaft that slides over the respective guide-wires, and forming a central hole concentric with each said circular depression to create two cavities to receive the pegs on the undersurface of the implant,
inserting a double-cannulated osteotome over said two guide-wires, which osteotome has a cutting blade that has two spaced apart opposite ends which are arcs that have radii respectively about equal to the radii of said two circles and cutting the bone at locations generally along two straight lines extending between and tangent to said two reamed circular depressions,
removing the osteotome and two generally triangular segments of bone from along the straight line cuts of the osteotome, and
locating the implant in the bone in which the depressions were reamed and securing it to the bone.

11. The method of claim 10 wherein each said central hole is respectively formed by a rotary cutting tool substantially simultaneously with the formation of each respective said circular depression.

12. The method of claim 10 wherein said two circular depressions have different diameters.

13. The method of claim 10 wherein the depth of cutting the bone by the osteotome is controlled by stops that extend outward beyond the opposite ends thereof and contact the bone surface.
